(19) United States
(12) Patent Application Publication
(10) Pub. No.: US 2005/0171482 A1
(43) Pub. Date: Aug. 4, 2005

(54) Medical tube holder with angled tabs

(76) Inventor: Ronald D. Russo, Barrington, RI (US)

Correspondence Address: FINNEGAN HENDERSON FARABOW;GARRETT & DUNNER LLP, 55 Cambridge Parkway, Cambridge, MA, 02142, US

(21) Appl. No.: 10/935,060

(22) Filed: Sep. 7, 2004

Related U.S. Application Data
(60) Provisional application No. 60/501,130, filed on Sep. 8, 2003.

(51) Int. Cl. A61M 5/32

(52) U.S. Cl. 604/180; 128/DIG. 26; 128/DIG. 6

(57) ABSTRACT

A medical tube holder is provided for securing and stabilizing medical tubes. In accordance with one illustrative embodiment, a medical tube holder is provided that includes a base for attachment to a patient's skin; a first tab extending from the base; and a second tab extending from the base at an angle with respect to the first tab greater than zero degrees and less than 180 degrees, wherein the first tab and second tab are adapted to each wrap around a medical tube in a spiral manner to secure the medical tube in place on the patient.

### RELATED APPLICATION

This application claims the benefit of U.S. Provisional Application, Ser. No. 60/501,130 filed Sep. 8, 2003, by Ronald D. Russo, entitled “Naso-Gastric Tube Holder,” which is hereby incorporated by reference in its entirety.

### BACKGROUND OF THE INVENTION

The use of medical tubes such as naso-gastric tubes and nasal airway tubes (hereinafter “nasal tubes”) is a common occurrence in medicine today. A naso-gastric tube is a tube fed through a patient's nostril, down the patient's esophagus, and into the patient's enteric tract (also called an “enteric tube”). A nasal airway tube is also fed through a patient's nostril, but rests in the tracheal airway, instead of extending down into the stomach.

One use of a naso-gastric tube is as a means of providing enteric nutrition (such as Ensure™) to a patient. The nutrition is delivered by the tube directly into the patient's stomach.

Another use of a naso-gastric tube is as a means of gastric decompression, used to drain gas and fluid from the stomach. Levin tubes, or Salem sump tubes, fall into this category. This type of tube relieves gas and fluid pressure resulting from trauma to the patient's enteric tract from an operation or injury.

Whether using a naso-gastric tube as a feeding tube or for gastric decompression, securing and stabilizing the tube is important. If not secured properly, lateral movement or awkward positioning of the tube can irritate the patients nostril nares, especially when the tube is in place for several days or weeks.

Retraction or advancement of the tube should also be prevented. Inadvertent retraction of the feeding tube can cause the tube to retreat to the esophagus where it can dispense fluid into the lungs. Inadvertent retraction of the gastric decompression tube can cause the tube to apply suction to sensitive esophageal tissues. Furthermore, proper positioning of the naso-gastric tube is often insured by taking an x-ray of the patient; thus, once properly positioned, it is a waste of time and resources to reposition the tube if not properly stabilized.

Commonly, naso-gastric tubes and other medical tubes are stabilized simply by taping the tube to the face of the patient. This often results in improper positioning of the tube, thereby irritating the nares. The direct application of medical tape to the patient's skin also tends to cause irritation, especially when removing the tape. Medical tape is also difficult and time consuming to apply and remove. Nasal airway tubes are also stabilized simply by taping the nose or face.

In addition to nasal tubes, there is also a need to secure and stabilize various medical tubes over other areas of a patient's body, e.g., intravenous tubes, gastronomy tubes, catheters, etc. The predominant means of securing and stabilizing most medical tubes is through the use of medical tape, whose disadvantages have been mentioned above.

### SUMMARY OF THE INVENTION

A medical tube holder is provided for securing and stabilizing medical tubes. In accordance with one illustrative embodiment, a medical tube holder is provided that includes a base for attachment to a patient's skin; a first tab extending from the base; and a second tab extending from the base at an angle with respect to the first tab greater than zero degrees and less than 180 degrees, wherein the first tab and second tab are adapted to each wrap around a medical tube in a spiral manner to secure the medical tube in place on the patient.

In accordance with another illustrative embodiment, a medical tube holder is provided that includes a base for attachment to a patient's skin; a first tab extending from the base; a second tab extending from the base, wherein the first tab and second tab are adapted to each wrap around a medical tube to secure the medical tube in place on the patient; and a release liner having a substantially greater surface area than the base, first tab, and second tab combined.

### BRIEF DESCRIPTION OF THE DRAWINGS

FIG. 1 is a top plan view of one illustrative embodiment of a medical tube holder consistent with principles of the present invention;

FIG. 2 is a rear plan view of the medical tube holder of FIG. 1; and

FIGS. 3 and 4 show the medical tube holder of FIGS. 1 and 2 applied to a patient to secure a nasal tube.

### DETAILED DESCRIPTION

Reference will now be made in detail to illustrative embodiments of the invention, an example of which is shown in the accompanying drawings. Wherever possible, the same reference numbers will be used throughout the drawings to refer to the same or like parts.

A medical tube holder is provided for securing medical tubes to any part of a patient's body. Such medical tubes include, but are not limited to, naso-gastric tubes, catheter tubes, levin tubes, salem sump tubes, intravenous tubes, etc. The tube holder has a unique unitary design having an adhesive on one side and two tabs extending from a base at an angle with respect to one another to facilitate securing and stabilizing a medical tube attached to a patient.

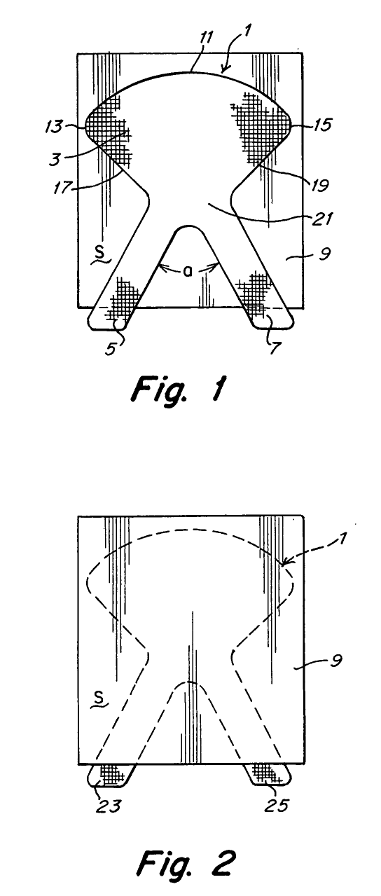

FIGS. 1 and 2 show one illustrative embodiment of a tube holder 1 consistent with principles of the present invention. The tube holder 1 includes a base 3 and first and second tabs 5 and 7. Base 3 is for adhering to a patient's skin and anchoring the tube holder, while tabs 5 and 7 are for securing the tube. To facilitate securing both the base 3 and tabs 5 and 7 to the patient and tube, respectively, an adhesive is applied to one side of the tube holder 1. As shown in FIGS. 1 and 2, this side, prior to application to a patient, is mounted on a release liner platform 9.

The tube holder 1 is constructed from a flexible and breathable material suitable for attachment to a patient's skin, and a heavy-duty fabric adhesive mounted on the material which adheres to the skin quickly and comfortably. For example, Bioflex® Rx715P from Scapa Tapes, North America, is an appropriate material. In the foregoing illustrative embodiment, the tube holder 1 is dye cut from Bioflex® Rx715P material, which includes an appropriate adhesive pre-applied thereon.

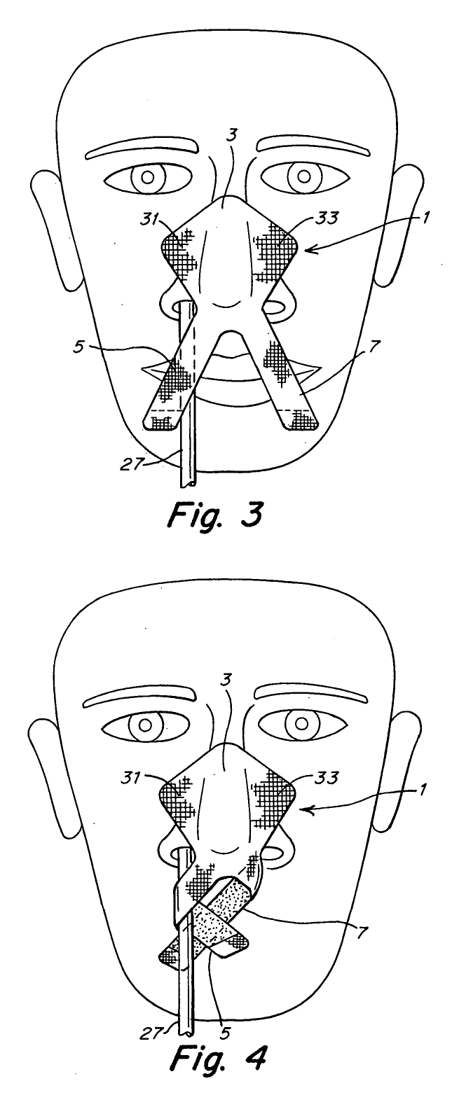

The base 3 has an irregular shape, including a semi-circular portion 11 which then curves inward at outermost points 13 and 15, and sides 17 and 19 which extend inwardly toward an intermediate portion 21 from which tabs 5 and 7 extend. Tabs 5 and 7 extend away from intermediate portion 21 at an angle “a”. Angle “a” is greater than 0° and less than 180°, more preferably between 10° and 170°, and still more preferably between 30° and 120°. In the foregoing illustrative embodiment an angle of approximately 60° is used. The use of an appropriate angle “a” facilitates wrapping tabs 5 and 7 about and securing the medical tube. The angled tabs enable each tab 5 and 7 to wrap about the tube in a “spiral” manner with one tab overlapping the other as shown in greater detail with reference to FIGS. 3 and 4 discussed below.

To facilitate both removal of the tube holder from the release liner platform 9 and attachment and detachment of the tabs 5 and 7 from the tube, ends 23 and 25 of tabs 5 and 7 are non-adherent (see FIG. 2). Thus, when the tube is secured, ends 23 and 25 each form a non-adherent lift edge, which permits easy removal by a caregiver with gloved hands. Non-adherent ends can be accomplished, for example, by simply not placing adhesive in these areas, or alternatively, permanently attaching a non-adhesive material, such as polyester, to ends 23 and 25. Polyester is an example of appropriate non-adherent material.

Release liner platform 9 is “cut” to have a substantial surface area “S”, in addition to the surface area covered by tube holder 1. This additional surface area “S” provides rigidity to the tube holder as well as a uniform shape (square in this example) which enables easier handling by the caregiver. The Bioflex® Rx715P material referred to above includes an appropriate release liner (4 mils 60 pound Kraft). Thus, to manufacture the tubeholder from a sheet of the Bioflex® Rx715P material, the tubeholder 1 is “kiss” cut from the material so as not to cut through the release liner, the release liner is cut in the desired shape surrounding the tubeholder, and the “excess” Bioflex® Rx715P material is removed from the surface area “S” of the release liner 9 surrounding the tube holder 1. The release liner 9 can also be pre-cut to facilitate removal thereof from the tube holder 1.

FIGS. 3 and 4 show the manner in which tube holder 1 is applied to a patient to secure a medical tube. In this example, the tube holder 1 is used to secure a naso-gastric tube 27 emanating from a patient's nostril. Tab 5 is wrapped over and around tube 27, while tab 7 is wrapped under and around tube 27. As shown in FIG. 4, the angling tabs 5 and 7 with respect to one anther enable each tab to be wrapped about tube 27 in a “spiral” manner. This provides the advantages of permitting the tabs grip the tube 27 along a greater surface area of the tube (down the length of the tube), and permits the overlapping tab (7 in FIG. 4) to both partially grip the tube 27 and the underlying tab (5 in FIG. 4), thereby providing additional securing and stabilizing force to the medical tube, as well as reinforcing attachment of the underlying tab. Also, the clockwise and counterclockwise arrangement of the tabs 5 and 7 work to strengthen the grip of each tab in that a force exerted on either tab will cause the other tab to tighten.

Alternatively, each tab 5 and 7 can be used to secure a separate tube, such as when a patient is intubated with both a Salem sump tube in one nostril, and a feeding tube in the second nostril. In such a case, each tab would wrap around each tube in a spiral manner as explained above, although no overlapping would occur because each tab is securing a separate tube.

It should be noted that the irregular shape of the base 3 of the illustrative embodiment of FIGS. 1-4 is especially useful in securing the base to a patient's nose, in that opposing sides 31 and 33 (FIGS. 3 and 4) are created that are conveniently shaped for attachment on either side of the patient's nose. It should be noted, however, that the base need not be the irregular shape as shown in FIGS. 1-4, and that other shapes may be used. For example, a circular base, an oval base, a square base, or a rectangular base can be used. In particular, such simpler shapes may be more appropriate for tubes that need securing on other parts of the patient's body, such as the arm or the leg.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A medical tube holder comprising:
a base for attachment to a patient's skin;
a first tab extending from the base; and
a second tab extending from the base at an angle with respect to the first tab greater than zero degrees and less than 180 degrees, wherein the first tab and second tab are adapted to each wrap around a medical tube in a spiral manner to secure the medical tube in place on the patient.

2. The medical tube holder of claim 1, wherein the base, first tab, and second tab are formed of a unitary material.

3. The medical tube holder of claim 1, wherein the base includes an adhesive for adhering to the patient's skin.

4. The medical tube holder of claim 1, wherein the first and second tabs include an adhesive for securing the medical tube.

5. The medical tube holder of claim 3, wherein the first and second tabs include an adhesive for securing the medical tube.

6. The medical tube holder of claim 5, wherein the base, first tab, and second tab are formed of a unitary material.

7. The medical tube holder of claim 1, wherein the base is an irregular shape to facilitate attachment to a patient's nose.

8. The medical tube holder of claim 1, wherein the base has one of a circular, oval, rectangular, and square shape.

9. The medical tube holder of claim 6, wherein the base is an irregular shape to facilitate attachment to a patient's nose.

10. The medical tube holder of claim 6, wherein the base has one of a circular, oval, rectangular, and square shape.

11. The medical tube holder of claim 4, wherein the tips of the first and second tabs are non-adherent, to facilitate removal of the tabs from a medical tube.

12. The medical tube holder of claim 6, wherein the tips of the first and second tabs are non-adherent, to facilitate removal of the tabs from a medical tube.

13. The medical tube holder of claim 11, wherein the tips include a non-adherent material attached thereto.

14. The medical tube holder of claim 13, wherein the non-adherent material is polyester.

15. The medical tube holder of claim 1, wherein the angle is between about 10 and about 170 degrees.

16. The medical tube holder of claim 1, wherein the angle is between about 30 and about 120 degrees.

17. The medical tube holder of claim 1, wherein the angle is about 60 degrees.

18. The medical tube holder of claim 1, wherein each tab is adapted to secure a separate medical tube.

19. The medical tube holder of claim 18, wherein each tab is adapted to secure a separate tube emanating from a respective nostril of a patient.

20. The medical tube holder of claim 1, wherein each tab is adapted to secure the same medical tube.

21. The medical tube holder of claim 1, wherein both the first and second tabs have non-adherent tips.

22. A medical tube holder comprising:
a base for attachment to a patient's skin;
a first tab extending from the base;
a second tab extending from the base, wherein the first tab and second tab are adapted to each wrap around a medical tube to secure the medical tube in place on the patient; and
a release liner having a substantially greater surface area than the surface area of the base, first tab, and second tab combined.

23. The medical tube holder of claim 22, wherein both the first and second tabs have non-adherent tips.

24. The medical tube holder of claim 22, wherein the second tab extends from the base at an angle with respect to the first tab greater than zero degrees and less than 180 degrees, wherein the first tab and second tab are adapted to each wrap around a medical tube in a spiral manner to secure the medical tube in place on the patient.

25. The medical tube holder of claim 23, wherein the second tab extends from the base at an angle with respect to the first tab greater than zero degrees and less than 180 degrees, wherein the first tab and second tab are adapted to each wrap around a medical tube in a spiral manner to secure the medical tube in place on the patient.
